(19) United States
(12) Patent Application Publication
(10) Pub. No.: US 2020/0118668 A1
(43) Pub. Date: Apr. 16, 2020

(54) METHOD AND APPARATUS FOR AUTISM SPECTRUM DISORDER ASSESSMENT AND INTERVENTION

(71) Applicant: INTERNATIONAL BUSINESS MACHINES CORPORATION, Armonk, NY (US)

(72) Inventors: Yi Qin Yu, Beijing (CN); Shi Lei Zhang, Beijing (CN); Jing Li, Beijing (CN); Wen Liu, Beijing (CN); Yong Qin, Beijing (CN)

(21) Appl. No.: 16/156,113

(22) Filed: Oct. 10, 2018

(51) Int. Cl. G16H 20/70; G16H 50/20

(52) U.S. Cl. CPC G16H 20/70; G16H 50/20

(57) ABSTRACT

A computer-implemented method, system, and computer program product are provided for Autism Spectrum Disorder assessment and intervention. The method includes receiving, by a processor device, behavioral phenomenon from a child. The method also includes generating, by the processor device, a similarity score for the child based on a similarity between the behavioral phenomenon and ASD profiles. The method additionally includes evaluating, by the processor device, the similarity score against applied behavior analysis (ABA) training courses. The method further includes determining, by the processor device, a dynamic ABA protocol from a sorted list of the ABA training courses. The method also includes controlling an operation of an interactive training device to deliver the dynamic ABA protocol to the child.

### BACKGROUND

### Technical Field

The present invention generally relates to Autism Spectrum Disorders, and more particularly to assessment and intervention for Autism Spectrum Disorders.

### Description of the Related Art

Autism Spectrum Disorders (ASD) are neurodevelopmental conditions characterized by persistent significant impairment in the social-communication domain along with restricted, repetitive patterns of behavior, interests and activities. While biological markers and specific causes for ASD have yet to be found, very early diagnosis and intervention are still the main approach to the condition.

### SUMMARY

In accordance with an embodiment of the present invention, a computer-implemented method is provided for Autism Spectrum Disorder assessment and intervention. The method includes receiving, by a processor device, behavioral phenomenon from a child. The method also includes generating, by the processor device, a similarity score for the child based on a similarity between the behavioral phenomenon and ASD profiles. The method additionally includes evaluating, by the processor device, the similarity score against applied behavior analysis (ABA) training courses. The method further includes determining, by the processor device, a dynamic ABA protocol from a sorted list of the ABA training courses. The method also includes controlling an operation of an interactive training device to deliver the dynamic ABA protocol to the child.

In accordance with another embodiment of the present invention, a computer program product is provided for Autism Spectrum Disorder assessment and intervention. The computer program product includes a non-transitory computer readable storage medium having program instructions. The program instructions are executable by a computer to cause the computer to perform a method. The method includes receiving, by a processor device, behavioral phenomenon from a child. The method also includes generating, by the processor device, a similarity score for the child based on a similarity between the behavioral phenomenon and ASD profiles. The method additionally includes evaluating, by the processor device, the similarity score against applied behavior analysis (ABA) training courses. The method further includes determining, by the processor device, a dynamic ABA protocol from a sorted list of the ABA training courses. The method also includes controlling an operation of an interactive training device to deliver the dynamic ABA protocol to the child.

In accordance with yet another embodiment of the present invention, an interactive training system is provided. The interactive training system includes a camera and a microphone. The interactive training system further includes a processing system having a processor device and memory receiving input from the camera and the microphone. The processing system is programmed to receive behavioral phenomenon from the camera and the microphone for a child. The processing system is also programmed to generate a similarity score for the child based on a similarity between the behavioral phenomenon and ASD profiles. The processing system is additionally programmed to evaluate the similarity score against applied behavior analysis (ABA) training courses. The processing system is further programmed to determine a dynamic ABA protocol from a sorted list of the ABA training courses. The processing system is also programmed to control an operation of the interactive training device to deliver the dynamic ABA protocol to the child.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 is an environment with an interactive training system utilizing a dynamic applied behavior analysis (ABA) system, in accordance with embodiments of the present invention;

FIG. 2 is a block/flow diagram of a dynamic ABA system, in accordance with embodiments of the present invention;

FIG. 3 is a block/flow diagram of an exemplary processing system with a dynamic ABA system, in accordance with embodiments of the present invention;

FIG. 4 is a block/flow diagram of an exemplary cloud computing environment, in accordance with an embodiment of the present invention;

FIG. 5 is a schematic diagram of exemplary abstraction model layers, in accordance with an embodiment of the present invention; and

FIG. 6 is a block/flow diagram of an Autism Spectrum Disorder assessment and intervention method, in accordance with an embodiment of the present invention.

### DETAILED DESCRIPTION

Embodiments in accordance with the present invention provide methods and apparatus for Autism Spectrum Disorder (ASD) assessment and intervention after a child has been diagnosed with ASD. Embodiments of the present invention can take audio and video based behaviors of the child as an input. Diverse audio and video technologies can be employed on the inputs to assess and depict the autistic characteristics of the child. Targeted intervention protocols can be generated based on the autistic characteristics of the child. The targeted interventions protocols can include synthesizing interactive talks corresponding to the autistic characteristics of the child.

Applied behavior analysis (ABA) is a scientific discipline concerned with applying techniques based upon the principles of learning to change behavior of social significance. Behaviors of social significance can include reading, academics, social skills, communication, and adaptive living skills. Adaptive living skills can include gross and fine motor skills, eating and food preparation, toileting, dressing, personal self-care, domestic skills, time and punctuality, money and value, home and community orientation, and work skills.

ABA training courses can increase behaviors (e.g., reinforcement procedures increase on-task behavior, or social interactions), teach new skills (e.g., systematic instruction and reinforcement procedures teach functional life skills, communication skills, or social skills), maintain behaviors (e.g., teaching self-control and self-monitoring procedures to maintain and generalize job-related social skills), generalize or transfer behavior from one situation or response to another (e.g., from completing assignments in the resource room to performing as well in the mainstream classroom), restrict or narrow conditions under which interfering behaviors occur (e.g., modifying the learning environment), and reduce interfering behaviors (e.g., self-injury or stereotypy).

A dynamic ABA system can analyze ABA training courses for multi-channel characteristics and the ASD specific behavioral phenomenon in the ABA training courses can be tagged. The ABA training courses can be designed around attention skills (sitting alone in the chair, responding when you hear the “put down” command, etc.), imitation skills (imitate the use of items, imitate fine movements, imitate lip movements, etc.), receptive language skills (response to naming, receive the certain command, point out drawings in the book, etc.) expressive language skills (call the name of certain things, follow me speaking, exchange greetings, etc.), and pre-academic skills (distinguish numbers and letters, distinguish the color, etc.), etc. The ABA training courses can include audio and/or video interaction with the child. The interaction can include a multitude of things. Examples of the interaction can include monitoring the child and responding, stimulate responses from the child, etc. Multi-channel characteristics can include acoustic characteristics or visual characteristics. Different acoustic characteristics can be collected, including low level characteristics, e.g., mel-frequency cepstral coefficients (MFCC), filter bank (Fbank), mel scale (MEL), and high level characteristics, e.g., energy, frequency, pitch. ASD specific behavioral phenomenon can be tagged based on the collected characteristics from the ABA training course. The ASD specific behavioral phenomenon can include challenging behaviors such as verbal protest (sensory overload-induced crying, screaming, shouting, and yelling), repeated speaking, etc.

The multi-channel characteristics can be clustered and aggregated for the ABA training course. The aggregation and clustering of the multi-channel characteristics can be accomplished with cluster analysis. Cluster analysis or clustering is the task of grouping a set of objects in such a way that objects in the same group (called a cluster) are more similar (in some sense) to each other than to those in other groups (clusters). Cluster analysis itself is not one specific algorithm, but a general task to be solved. The dynamic ABA system can utilize various cluster analysis algorithms for the clustering and aggregating of the multi-channel characteristics. In one embodiment, the dynamic ABA system can employ hierarchical agglomerative clustering, which builds clusters following a “bottom up” approach. The aggregated characteristics may reflect the characteristics of ASD behaviors, such as Asperger's, Rett Syndrome, etc. The aggregated characteristics can be employed to identify which ASD specific behaviors can be helped with the ABA training courses. The aggregated characteristics can be utilized to generate a characteristic vector for each of the ABA training courses. The characteristic vectors can be clustered to form ASD profiles. Each cluster of vectors can have a relationship between the ABA training course and an evaluation score. An example of the evaluation score can be Psycho-educational Profile 3 score (PEP-3). A PEP-3 score can be attained from a teacher evaluating a child. The relationship between the ABA training course and the evaluation score can be described as XWᵢ=Y, where X is a list of the ABA training courses, Wᵢ is the weight between X and Y in the i^{th} cluster, and Y is a list of the evaluation scores.

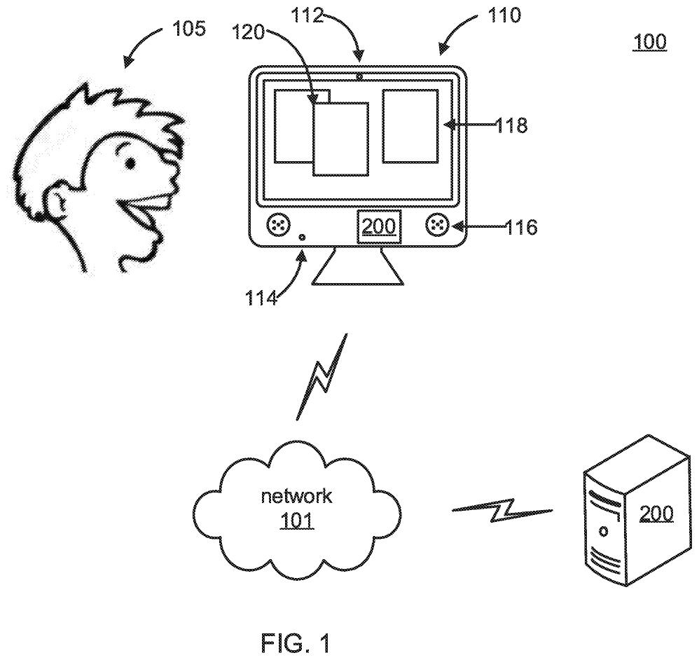

Referring now to the drawings in which like numerals represent the same or similar elements and initially to FIG. 1, an environment 100 to which the present invention may be applied is shown in accordance with one embodiment. The environment 100 can include an interactive training system 110 with a dynamic ABA system 200. In one embodiment, the interactive training system 110 can have the dynamic ABA system 200 integrated into the interactive training system 110. In another embodiment, the dynamic ABA system can be a remote system connected to the interactive training system 110 through a network 101. The interactive training system 110 can include a camera 112 and a microphone 114. The camera 112 and the microphone 114 can capture audio and video data of a new ASD child 105. The interactive training system can include speakers 116 and a display 118. The speakers 116 play material to the new ASD child 105 and the display 118 can show the new ASD child 105 material. The material can include ABA training courses 120.

The ABA training courses 120 relationships to ASD profiles can be utilized when the new ASD child 105 is being treated. The new ASD child 105 can be tested for behavioral phenomenon so the similarity between the new ASD child 105 and all ASD profiles can be calculated to get a similar score, S. The importance of utilizing an ABA training course 120 in the ABA training courses list, xᵢ in X, can be evaluated for the new ASD child 105 with ΣWᵢ*Sᵢ. All of the courses in the ABA training courses list can be sorted according to importance for the new ASD child 105 to build a dynamic ABA protocol for the new ASD child 105. The dynamic ABA protocol can include several of the ABA training courses 120, e.g., x₁, x₅, x₇₆ as an example. The new ASD child 105 can start with the dynamic ABA protocol utilizing an interactive training system 110. Examples of interactive training systems 110 include computers, tablets, other mobile devices, robots, etc. The interactive training system 110 can utilize the ABA training courses 120 in the dynamic ABA protocol with the new ASD child 105. The interactive training system 110 can continually monitor the new ASD child 105 while administering the ABA training courses 120 to update the similar score of the new ASD child 105 to update the dynamic ABA protocol to add or remove ABA training courses 120.

Costs for ASD training and intervention can be very high due to long and labor intensive work by professionals. The dynamic ABA protocols described above can benefit every ASD child by saving cost and providing a more personalized and dynamic intervention protocol automatically.

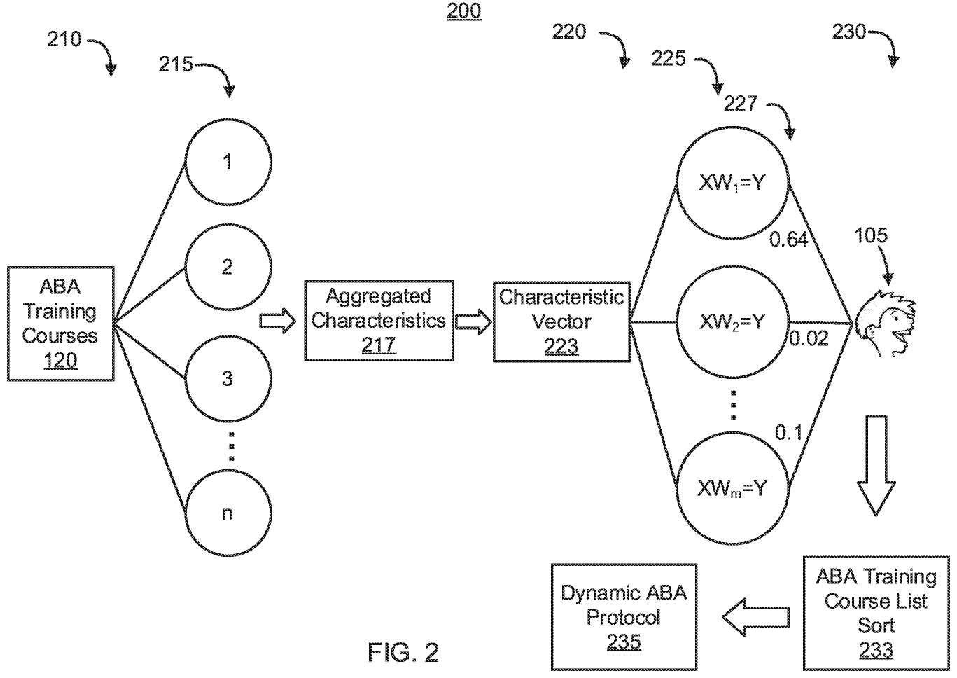

FIG. 2 is block/flow diagram of a dynamic ABA system 200, in accordance with embodiments of the present invention.

The dynamic ABA system 200 can aggregate ABA protocol characteristics 210. The aggregation of ABA protocol characteristics 210 can include analyzing ABA training courses 120 for multi-channel characteristics 215 with the ASD specific behavioral phenomenon in the ABA training courses 120 being tagged. Multi-channel characteristics 215 can include acoustic characteristics or visual characteristics. ASD specific behavioral phenomenon can be tagged based on the collected characteristics from the ABA training courses 120. The multi-channel characteristics 215 can be clustered and aggregated into aggregated characteristics 217. The aggregated characteristics 217 can be utilized in an ASD profile buildup 220.

The ASD profile buildup 220 can employ the aggregated characteristics 217 to identify which ASD specific behaviors can be helped with the ABA training courses 120. The aggregated characteristics 217 can be utilized to generate a characteristic vector 223 for each of the ABA training courses 120. The characteristic vectors 223 can be clustered to form ASD profiles 225. Each ASD profile 225 can have a relationship between the ABA training course 120 and an evaluation score. The relationship can be described as XWᵢ=Y, where X is a list of the ABA training courses 120, Wᵢ is the weight between X and Y in the i^{th} cluster, and Y is a list of the evaluation scores. The ASD profiles 225 can be utilized to generate a dynamic ABA protocol suggestion 230.

The dynamic ABA protocol suggestion 230 can employ the ASD profiles 225 when a new ASD child 105 begins treatment. The new ASD child 105 can be tested so the similarity between the new ASD child 105 and all ASD profiles 225 can be calculated to get a similar score 227. The Similar score 227 can range from zero to one, examples can include 0.64, 0.02, and 0.1. The importance of utilizing an ABA training course 120 in the ABA training courses list, xᵢ in X, can be evaluated for the new ASD child 105 with ΣWᵢ*Sᵢ. All of the courses in the ABA training courses list can be sorted 233 according to the courses importance for the new ASD child 105 in order to build a dynamic ABA protocol 235 for the new ASD child 105. The dynamic ABA protocol 235 can include several of the ABA training courses 120, e.g., x₆, x₃, x₁₅ as an example.

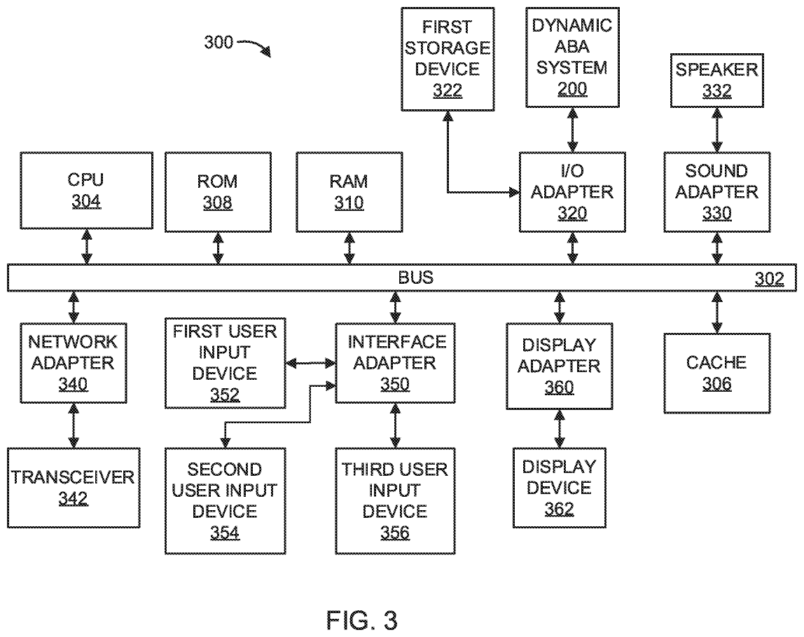

FIG. 3 is an exemplary processing system 300 with a dynamic ABA system 200, in accordance with an embodiment of the present invention. The processing system 300 includes at least one processor (CPU) 304 operatively coupled to other components via a system bus 302. A cache 306, a Read Only Memory (ROM) 308, a Random Access Memory (RAM) 310, an input/output (I/O) adapter 320, a sound adapter 330, a network adapter 340, a user interface adapter 350, and a display adapter 360, are operatively coupled to the system bus 302.

A first storage device 322 is operatively coupled to system bus 302 by the I/O adapter 320. The storage device 322 can be any of a disk storage device (e.g., a magnetic or optical disk storage device), a solid state magnetic device, and so forth. The dynamic ABA system 200 can be coupled to the system bus 302 by the I/O adapter 320. The dynamic ABA system 200 can exchange audio and video data with the processing system 300. The exchange can include sending audio or video data to be played by the processing system 300 or receiving audio or video data the processing system 300 is detecting.

A speaker 332 is operatively coupled to system bus 302 by the sound adapter 330. A transceiver 342 is operatively coupled to system bus 302 by network adapter 340. A display device 362 is operatively coupled to system bus 302 by display adapter 360.

A first user input device 352, a second user input device 354, and a third user input device 356 are operatively coupled to system bus 302 by user interface adapter 350. The user input devices 352, 354, and 356 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 352, 354, and 356 can be the same type of user input device or different types of user input devices. The user input devices 352, 354, and 356 are used to input and output information to and from system 300.

Of course, the processing system 300 may also include other elements (not shown), as readily contemplated by one of skill in the art, as well as omit certain elements. For example, various other input devices and/or output devices can be included in processing system 300, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 300 are readily contemplated by one of ordinary skill in the art given the teachings of the present invention provided herein.

Moreover, it is to be appreciated that environment 100 described above with respect to FIG. 1 is an environment for implementing respective embodiments of the present invention. Part or all of processing system 300 may be implemented in one or more of the elements of environment 100.

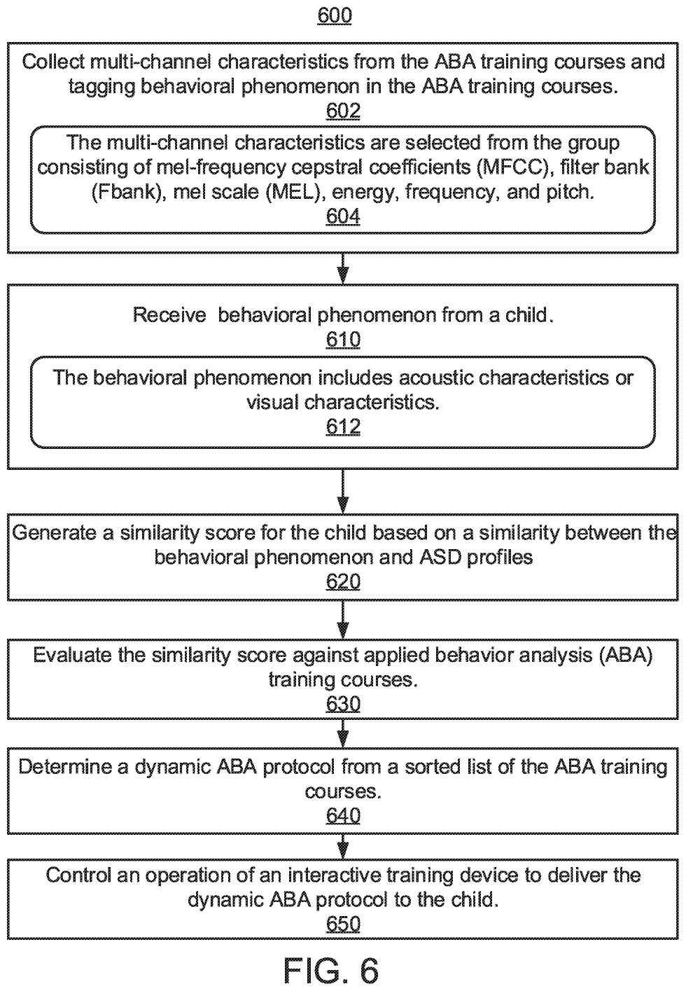

Further, it is to be appreciated that processing system 300 may perform at least part of the method described herein including, for example, at least part of the interactive training system 110 of FIG. 1 and/or at least part of method 600 of FIG. 6.

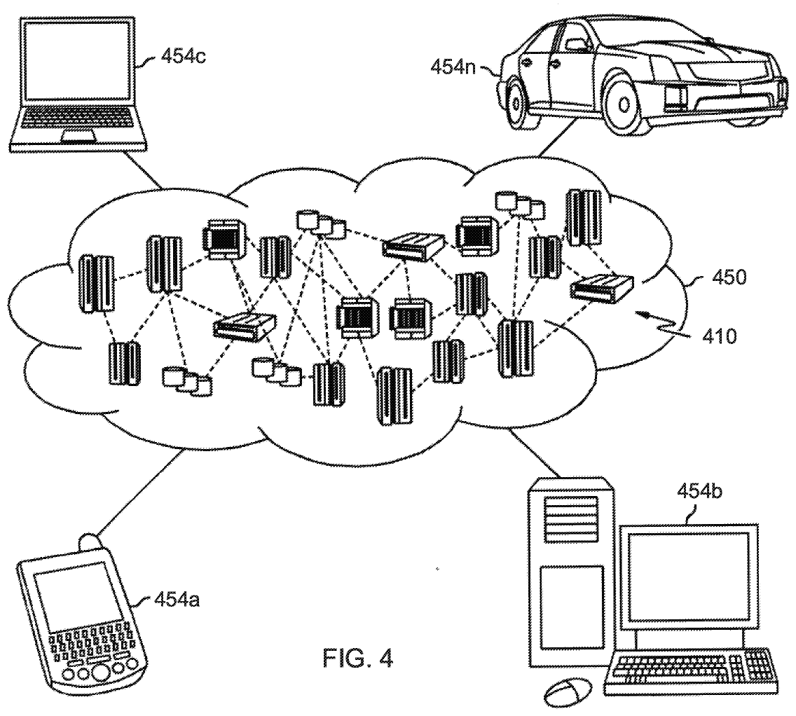

FIG. 4 is a block/flow diagram of an exemplary cloud computing environment, in accordance with an embodiment of the present invention.

It is to be understood that although this invention includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model can include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but can be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It can be managed by the organization or a third party and can exist on-premises or off-premises.

Community cloud: the cloud infrastructure is shared by several organizations and supports a specific community that has shared concerns (e.g., mission, security requirements, policy, and compliance considerations). It can be managed by the organizations or a third party and can exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 450 is depicted for enabling use cases of the present invention. As shown, cloud computing environment 450 includes one or more cloud computing nodes 410 with which local computing devices used by cloud consumers, such as, for example, personal digital assistant (PDA) or cellular telephone 454A, desktop computer 454B, laptop computer 454C, and/or automobile computer system 454N can communicate. Nodes 410 can communicate with one another. They can be grouped (not shown) physically or virtually, in one or more networks, such as Private, Community, Public, or Hybrid clouds as described hereinabove, or a combination thereof. This allows cloud computing environment 450 to offer infrastructure, platforms and/or software as services for which a cloud consumer does not need to maintain resources on a local computing device. It is understood that the types of computing devices 454A-N shown in FIG. 4 are intended to be illustrative only and that computing nodes 410 and cloud computing environment 450 can communicate with any type of computerized device over any type of network and/or network addressable connection (e.g., using a web browser).

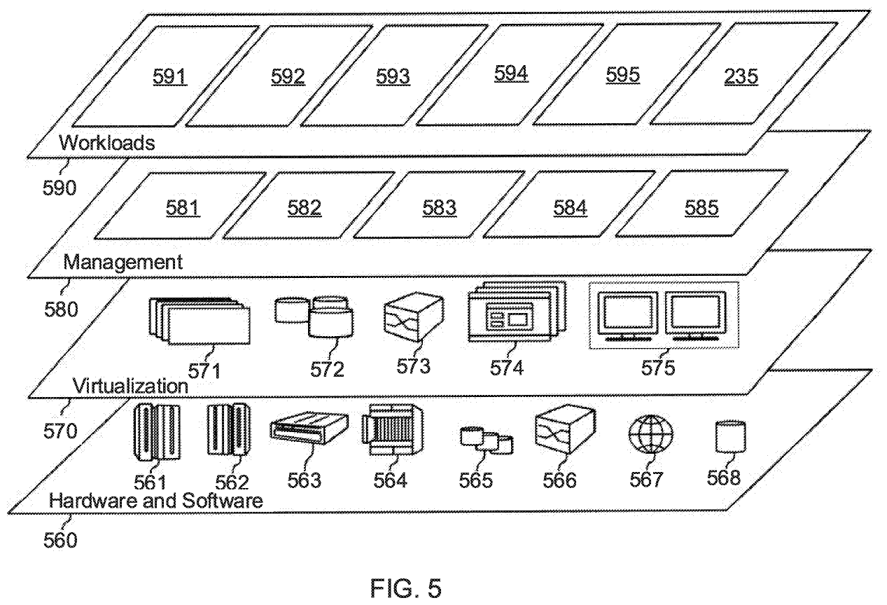

FIG. 5 is a schematic diagram of exemplary abstraction model layers, in accordance with an embodiment of the present invention. It should be understood in advance that the components, layers, and functions shown in FIG. 5 are intended to be illustrative only and embodiments of the invention are not limited thereto. As depicted, the following layers and corresponding functions are provided:

Hardware and software layer 560 includes hardware and software components. Examples of hardware components include: mainframes 561; RISC (Reduced Instruction Set Computer) architecture based servers 562; servers 563; blade servers 564; storage devices 565; and networks and networking components 566. In some embodiments, software components include network application server software 567 and database software 568.

Virtualization layer 570 provides an abstraction layer from which the following examples of virtual entities can be provided: virtual servers 571; virtual storage 572; virtual networks 573, including virtual private networks; virtual applications and operating systems 574; and virtual clients 575.

In one example, management layer 580 can provide the functions described below. Resource provisioning 581 provides dynamic procurement of computing resources and other resources that are utilized to perform tasks within the cloud computing environment. Metering and Pricing 582 provide cost tracking as resources are utilized within the cloud computing environment, and billing or invoicing for consumption of these resources. In one example, these resources can include application software licenses. Security provides identity verification for cloud consumers and tasks, as well as protection for data and other resources. User portal 583 provides access to the cloud computing environment for consumers and system administrators. Service level management 584 provides cloud computing resource allocation and management such that required service levels are met. Service Level Agreement (SLA) planning and fulfillment 585 provide pre-arrangement for, and procurement of, cloud computing resources for which a future requirement is anticipated in accordance with an SLA.

Workloads layer 590 provides examples of functionality for which the cloud computing environment can be utilized. Examples of workloads and functions which can be provided from this layer include: mapping and navigation 591; software development and lifecycle management 592; virtual classroom education delivery 593; data analytics processing 594; transaction processing 595; and the dynamic ABA protocol 235.

Referring to FIG. 6, a flow chart for an Autism Spectrum Disorder assessment and intervention method 600 is illustratively shown, in accordance with an embodiment of the present invention. In block 602, multi-channel characteristics are collected from the ABA training courses and behavioral phenomenon are tagged in the ABA training courses. In block 604, the multi-channel characteristics are selected from the group consisting of mel-frequency cepstral coefficients (MFCC), filter bank (Fbank), mel scale (MEL), energy, frequency, and pitch. In block 610, behavioral phenomenon from a child is received. In block 612, the behavioral phenomenon includes acoustic characteristics or visual characteristics. In block 620, a similarity score is generated for the child based on a similarity between the behavioral phenomenon and ASD profiles. In block 630, the similarity score is evaluated against applied behavior analysis (ABA) training courses. In block 640, a dynamic ABA protocol is determined from a sorted list of the ABA training courses. In block 650, an operation of an interactive training device is controlled to deliver the dynamic ABA protocol to the child.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A computer-implemented method for Autism Spectrum Disorder assessment and intervention, the method comprising:
receiving, by a processor device, behavioral phenomenon from a child;
generating, by the processor device, a similarity score for the child based on a similarity between the behavioral phenomenon and ASD profiles;
evaluating, by the processor device, the similarity score against applied behavior analysis (ABA) training courses;
determining, by the processor device, a dynamic ABA protocol from a sorted list of the ABA training courses; and
controlling an operation of an interactive training device to deliver the dynamic ABA protocol to the child.

2. The computer-implemented method as recited in claim 1, wherein receiving includes receiving behavioral phenomenon that includes acoustic characteristics or visual characteristics.

3. The computer-implemented method as recited in claim 1, further comprises collecting multi-channel characteristics from the ABA training courses and tagging behavioral phenomenon in the ABA training courses.

4. The computer-implemented method as recited in claim 3, wherein collecting includes identifying characteristics selected from the group consisting of mel-frequency cepstral coefficients (MFCC), filter bank (Fbank), mel scale (MEL), energy, frequency, and pitch.

5. The computer-implemented method as recited in claim 1, wherein receiving includes receiving behavioral phenomenon selected from the group consisting of verbal protests and repeated speaking.

6. The computer-implemented method as recited in claim 1, further comprises aggregating and clustering multi-channel characteristics for the ABA training courses to form aggregated characteristics for the ABA training courses.

7. The computer-implemented method as recited in claim 1, further comprises generating a characteristic vector for each of the ABA training courses utilizing aggregated characteristics for each of the ABA training courses.

8. The computer-implemented method as recited in claim 1, further comprises clustering characteristic vectors of the ABA training courses to form the ASD profiles

9. The computer-implemented method as recited in claim 1, wherein the ASD profiles include a relationship between the ABA training course and an evaluation score.

10. The computer-implemented method as recited in claim 9, wherein the evaluation score includes a Psycho-educational Profile 3 score.

11. The computer-implemented method as recited in claim 1, further comprises monitoring the child during deployment of the dynamic ABA protocol and updating the dynamic ABA protocol responsive to responses of the child to the dynamic ABA protocol.

12. A computer program product for Autism Spectrum Disorder assessment and intervention, the computer program product comprising a non-transitory computer readable storage medium having program instructions embodied therewith, the program instructions executable by a computer to cause the computer to perform a method comprising:
receiving, by a processor device, behavioral phenomenon from a child;
generating, by the processor device, a similarity score for the child based on a similarity between the behavioral phenomenon and ASD profiles;
evaluating, by the processor device, the similarity score against applied behavior analysis (ABA) training courses;
determining, by the processor device, a dynamic ABA protocol from a sorted list of the ABA training courses; and
controlling an operation of an interactive training device to deliver the dynamic ABA protocol to the child.

13. An interactive training system for Autism Spectrum Disorder assessment and intervention, comprising:
a camera and a microphone;
a processing system including a processor device and memory receiving input from the camera and the microphone, the processing system programmed to:
receive behavioral phenomenon from the camera and the microphone for a child;
generate a similarity score for the child based on a similarity between the behavioral phenomenon and ASD profiles;
evaluate the similarity score against applied behavior analysis (ABA) training courses;
determine a dynamic ABA protocol from a sorted list of the ABA training courses; and
control an operation of the interactive training device to deliver the dynamic ABA protocol to the child.

14. The system as recited in claim 13, wherein the behavioral phenomenon includes acoustic characteristics or visual characteristics.

15. The system as recited in claim 13, further programmed to collect multi-channel characteristics from the ABA training courses and tag behavioral phenomenon in the ABA training courses.

16. The system as recited in claim 15, wherein the multi-channel characteristics are selected from the group consisting of mel-frequency cepstral coefficients (MFCC), filter bank (Fbank), mel scale (MEL), energy, frequency, and pitch.

17. The system as recited in claim 13, wherein the behavioral phenomenon is selected from the group consisting of verbal protests and repeated speaking.

18. The system as recited in claim 13, further programmed to aggregate and cluster multi-channel characteristics for the ABA training courses to form aggregated characteristics for the ABA training courses.

19. The system as recited in claim 13, further programmed to generate a characteristic vector for each of the ABA training courses utilizing aggregated characteristics for each of the ABA training courses.

20. The system as recited in claim 13, further programmed to form the ASD profiles by clustering characteristic vectors of the ABA training courses.
